(19) United States
(12) Patent Application Publication
(10) Pub. No.: US 2017/0131291 A1
(43) Pub. Date: May 11, 2017

(54) METHODS AND DEVICES FOR DIAGNOSING OCULAR SURFACE INFLAMMATION AND DRY EYE DISEASE

(71) Applicant: Seinda Biomedical Corporation

(72) Inventor: Jing-Feng HUANG, San Diego, CA (US)

(21) Appl. No.: 15/285,842

(22) Filed: Oct. 5, 2016

Related U.S. Application Data
(60) Provisional application No. 62/237,488, filed on Oct. 5, 2015.

(51) Int. Cl. G01N 33/68

(52) U.S. Cl. CPC G01N 33/6869; G01N 33/6863; G01N 2800/16; G01N 2800/245; G01N 2800/24; G01N 2800/52; G01N 2800/50; G01N 2333/5406; G01N 2333/5403; G01N 2333/535; G01N 2333/5428

(57) ABSTRACT

The present invention relates to the diagnosis, monitoring, and/or treatment of medical conditions and/or a predisposition thereto. The condition preferably is dry eye disease. Described herein are methods, kits, and devices for diagnosing and monitoring dry eye disease in a subject.

### RELATED APPLICATION

This patent application claims the benefit of and priority to U.S. provisional patent application Ser. No. 62/237,494, filed on 5 Oct. 2015, which is hereby incorporated in its entirety for any and all purposes.

### TECHNICAL FIELD

This invention concerns devices, kits, and methods for diagnosing dye eye and related ocular surface diseases, as well as to methods of using data and information generated through the use of such devices, kits, and methods.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

The ocular surface is continuously exposed to environmental agents such as allergens, pollutants, and microorganisms, which could provoke inflammation. The cornea and the underlying anterior chamber possess unique attributes that protect the cornea and the eye from immune-mediated inflammation and immune-mediated injury in the eye and create ocular immune tolerance, which is believed to be essential for maintaining normal vision and a healthy eye.

Dry eye disease (DED) is one of the most prevalent eye conditions, affecting millions of people in the United States alone and many millions more in other regions of the world. Ocular symptoms often experienced by dry eye patients include dryness, ocular irritation and/or pain, and blurred vision, which can significantly affect quality of life and work-related activities. DED patients also report greater sensitivities and less tolerance to changes in their environments. Visual dysfunction includes difficulty in reading, driving, computer usage, watching TV, and other daily personal and work-related activities. Diagnosis of dry eye disease is typically based on subjective symptoms, tear break-up time (evaluating quality of tear film), vital dye staining of the ocular surface such as corneal fluorescein staining, the Schirmer test (evaluating quantity of tear fluid), and other less common clinical tests, including tear osmolarity, Rose Bengal staining, measuring tear meniscus height, and others. Numerous studies have shown that correlation is poor between clinical tests and symptoms and even between different clinical tests. Common pathological changes occurred in dry eye include decreased goblet cell density, reduced mucin production, increased apoptosis, and epithelial squamous metaplasia.

Efforts in delineating underlying inflammatory mechanism and pathways in DED have led to greater understanding of the role of inflammation in disease pathogenesis. However, poor precision in DED clinical measurements, lack of a “gold standard” for defining disease severity, discordance between patient-reported ocular symptoms and clinical signs, and between different clinical parameters, and significant patient heterogeneity have been major challenges in DED clinical research and clinical development of therapeutic treatments. Thus, there still exists a significant need in developing improved, reliable, objective, robust, and sensitive methods, reagents, and tools that could be used for diagnosis and monitoring of DED and for monitoring treatment of DED.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

The term “risk” relates to the possibility or probability of a particular event occurring either presently, or, at some point in the future. “Risk stratification” refers to an arraying of known clinical risk factors to allow physicians to classify patients into a low, moderate, high or highest risk of developing of a particular disease, disorder, or condition.

“Diagnosing” includes determining, monitoring, confirmation, subclassification, and prediction of the relevant disease, complication, or risk. “Determining” relates to becoming aware of a disease, complication, risk, or entity (e.g., biomarker). “Monitoring” relates to keeping track of an already diagnosed disease, complication, or risk factor, e.g., to analyze the progression of the disease or the influence of a particular treatment on the progression of disease or complication. “Confirmation” relates to the strengthening or substantiating of a diagnosis already performed using other indicators or markers. “Classification” or “subclassification” relates to further defining a diagnosis according to different subclasses of the diagnosed disease, disorder, or condition, e.g., defining according to mild, moderate, or severe forms of the disease or risk. “Prediction” relates to prognosing a disease, disorder, condition, or complication before other symptoms or markers have become evident or have become significantly altered.

A “subject” is a member of any animal species, preferably a mammalian species, optionally a human. Thus, the methods, compositions, reagents, and kits described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology. The subject can be an apparently healthy individual, an individual suffering from a disease, or an individual being treated for a disease. A “reference subject” or “reference subjects” is/are an individual or a population that serves as a reference against which to assess another individual or population with respect to one or more parameters.

The term “normal” or “clinically normal” means the subject has no known or apparent or presently detectable disease or dysfunction and no detectable increase or decrease in biomarkers associated with dry eye disease.

“Samples” that can be assayed using the methods of the present invention include biological fluids, such as whole blood, serum, plasma, tear, saliva, synovial fluid, cerebrospinal fluid, bronchial lavage, ascites fluid, bone marrow aspirate, pleural effusion, urine, as well as tumor tissue or any other bodily constituent or any tissue culture supernatant that could contain the analyte of interest. Samples can be obtained by any appropriate method known in the art.

An “analyte” refers to the substance to be detected, which may be suspected of being present in the sample (i.e., the biological sample). The analyte can be any substance for which there exists a naturally occurring specific binding partner or for which a specific binding partner can be prepared. Thus, an analyte is a substance that can bind to one or more specific binding partners in an assay.

A “binding partner” is a member of a binding pair, i.e., a pair of molecules wherein one of the molecules binds to the second molecule. Binding partners that bind specifically are termed “specific binding partners.” In addition to antigen and antibody binding partners commonly used in immunoassays, other specific binding partners can include biotin and avidin (or streptavidin), carbohydrates and lectins, nucleic acids with complementary nucleotide sequences, effector and receptor molecules, cofactors and enzymes, enzyme inhibitors and enzymes, and the like. Furthermore, specific binding partners can include partner(s) that is/are analog(s) of the original specific binding partner, for example, an analyte-analog. Immunoreactive specific binding partners include antigens, antigen fragments, antibodies and antibody fragments, both monoclonal and polyclonal, and complexes thereof, including those formed by recombinant DNA methods.

As used herein, the term “epitope” or “epitopes,” or “epitopes of interest” refer to a site(s) on any molecule that is recognized and is capable of binding to a complementary site(s) on its specific binding partner. The epitope-bearing molecule and specific binding partner are part of a specific binding pair. For example, an epitope can be a polypeptide, protein, hapten, carbohydrate antigen (such as, but not limited to, glycolipids, glycoproteins or lipopolysaccharides) or polysaccharide and its specific binding partner, can be, but is not limited to, an antibody. Typically an epitope is contained within a larger molecular framework (e.g., in the context of an antigenic region of a protein, the epitope is the region or fragment of the protein having the structure capable of being bound by an antibody reactive against that epitope) and refers to the precise residues known to contact the specific binding partner. As is known, it is possible for an antigen or antigenic fragment to contain more than one epitope.

As used herein, “specific” or “specificity” in the context of an interaction between members of a specific binding pair (e.g., an antigen and antibody) refers to the selective reactivity of the interaction. The phrase “specifically binds to” and analogous terms thereof refer to the ability of antibodies to specifically bind to (e.g., preferentially react with) an endogenous antigen and not specifically bind to other entities. Antibodies (including autoantibodies) or antibody fragments that specifically bind to an endogenous antigen correlated with dry eye disease can be identified, for example, by diagnostic immunoassays (e.g., radioimmunoassays (“RIA”) and enzyme-linked immunosorbent assays (“ELISAs”), surface plasmon resonance, or other techniques known to those of skill in the art. In one embodiment, the term “specifically binds” or “specifically reactive” indicates that the binding preference (e.g., affinity) for the target analyte is at least about 2-fold, more preferably at least about 5-fold, 10-fold, 100-fold, 1,000-fold, a million-fold or more over a non-specific target molecule (e.g., a randomly generated molecule lacking the specifically recognized site(s)).

An antigen, biomarker, or other analyte “correlated” or “associated” with a disease, particularly dry eye disease, refers to a biomarker or other analyte that is positively correlated with the presence or occurrence of dry eye disease generally or a specific dry eye disease, as the context requires. In general, an “antigen” is any substance that exhibits specific immunological reactivity with a target antibody. Suitable antigens, particularly biomarkers, may include, without limitation, molecules comprising at least one antigenic epitope capable of interacting specifically with the variable region or complementarity determining region (CDR) of an antibody or CDR-containing antibody fragment. Antigens typically are naturally occurring or synthetic biological macromolecules such as a protein, peptide, polysaccharide, lipids, or nucleic acids, or complexes containing these or other molecules.

As used herein with reference to a disease-associated antigens (or other analytes correlated with dry eye disease), the term “elevated level” refers to a level in a sample that is higher than a normal level or range, or is higher that another reference level or range (e.g., earlier or baseline sample). The term “altered level” refers to a level in a sample that is altered (increased or decreased) over a normal level or range, or over another reference level or range (e.g., earlier or baseline sample). The normal level or range for a particular biomarker is defined in accordance with standard practice. Because the levels of biomarkers in some instances will be very low, a so-called altered level or alteration can be considered to have occurred when there is any net change as compared to the normal level or range, or reference level or range that cannot be explained by experimental error or sample variation. Thus, the level measured in a particular sample will be compared with the level or range of levels determined in similar samples of normal tissue. In this context, “normal tissue” is tissue from an individual with no detectable dry eye pathology, and a “normal” (sometimes termed “control”) patient (i.e., subject) or population is one that exhibits no detectable pathology. The level of an analyte is said to be “elevated” where the analyte is normally undetectable (e.g., the normal level is zero, or within a range of from about 25 to about 75 percentiles of normal populations), but is detected in a test sample, as well as where the analyte is present in the test sample at a higher than normal level.

An “array” refers a device consisting of a substrate, typically a solid support having a surface adapted to receive and immobilize a plurality of different protein, peptide, and/or nucleic acid species (i.e., capture or detection reagents) that can used to determine the presence and/or amount of other molecules (i.e., analytes) in biological samples such as blood. A “microarray” refers to an array wherein the different detection reagents disposed on the substrate in a grid or other pattern.

The term “solid phase” refers to any material or substrate that is insoluble, or can be made insoluble by a subsequent reaction. A solid phase can be chosen for its intrinsic ability to attract and immobilize a capture or detection reagent. Alternatively, a solid phase can have affixed thereto a linking agent that has the ability to attract and immobilize a capture agent. The linking agent can, for example, include a charged substance that is oppositely charged with respect to the capture agent itself or to a charged substance conjugated to the capture agent. In general, a linking agent can be any binding partner (preferably specific) that is immobilized on (said to be “attached to”) a solid phase and that has the ability to immobilize a desired capture or detection reagent through a binding or other associative reaction. A linking agent enables the indirect binding of a capture agent to a solid phase material before the performance of an assay or during the performance of an assay. The solid phase can, for example, be plastic, derivatized plastic, magnetic or non-magnetic metal, glass or silicon, including, for example, a test tube, microtiter well, sheet, bead, microparticle, chip, and other configurations known to those of ordinary skill in the art.

As used herein, term “microparticle” refers to a small particle that is recoverable by any suitable process, e.g., magnetic separation or association, ultracentrifugation, etc. Microparticles typically have an average diameter on the order of about 1 micron or less.

A “capture” or “detection” agent or reagent refers to a binding partner that binds to an analyte, preferably specifically. Capture or detection reagents can be attached to or otherwise associated with a solid phase.

The term “labeled detection agent” refers to a binding partner that binds to an analyte, preferably specifically, and is labeled with a detectable label or becomes labeled with a detectable label during use in an assay. A “detectable label” includes a moiety that is detectable or that can be rendered detectable. With reference to a labeled detection agent, a “direct label” is a detectable label that is attached, by any means, to the detection agent, and an “indirect label” is a detectable label that specifically binds the detection agent. Thus, an indirect label includes a moiety that is the specific binding partner of a moiety of the detection agent. Biotin and avidin are examples of such moieties that can be employed, for example, by contacting a biotinylated antibody with labeled avidin to produce an indirectly labeled antibody.

The term “indicator reagent” refers to any agent that is contacted with a label to produce a detectable signal. Thus, for example, in conventional enzyme labeling, an antibody labeled with an enzyme can be contacted with a substrate (the indicator reagent) to produce a detectable signal, such as a colored reaction product.

An “antibody” refers to a protein consisting of one or more polypeptides substantially encoded by immunoglobulin genes or fragments of immunoglobulin genes. This term encompasses polyclonal antibodies, monoclonal antibodies, and fragments thereof, as well as molecules engineered from immunoglobulin gene sequences. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Antibodies are generally found in bodily fluids, mainly blood.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains, respectively.

Antibodies exist as intact immunoglobulins or as a number of well-characterized fragments produced by digestion with various peptidases. Thus, for example, pepsin digests an antibody below the disulfide linkages in the hinge region to produce F(ab′)₂, a dimer of Fab which itself is a light chain joined to VH-CH1 by a disulfide bond. The F(ab′)₂ may be reduced under mild conditions to break the disulfide linkage in the hinge region thereby converting the (Fab′)₂ dimer into a Fab′ monomer. The Fab′ monomer is essentially a Fab with part of the hinge region. While various antibody fragments are defined in terms of the digestion of an intact antibody, one of skill will appreciate that such Fab′ fragments may be synthesized de novo either chemically or by utilizing recombinant DNA methodology. Thus, in the context of the invention the term “antibody” also includes antibody fragments either produced by the modification of whole antibodies or synthesized de novo using recombinant DNA methodologies. Antibodies include single chain antibodies (antibodies that exist as a single polypeptide chain), single chain Fv antibodies (sFv or scFv), in which a variable heavy and a variable light chain are joined together (directly or through a peptide linker) to form a continuous polypeptide. The single chain Fv antibody is a covalently linked VH-VL heterodimer that may be expressed from a nucleic acid including VH- and VL-encoding sequences either joined directly or joined by a peptide-encoding linker. While the VH and VL are connected to each as a single polypeptide chain, the VH and VL domains associate non-covalently. The scFv antibodies and a number of other structures convert the naturally aggregated, but chemically separated, light and heavy polypeptide chains from an antibody V region into a molecule that folds into a three dimensional structure substantially similar to the structure of an antigen-binding site are known to those of skill in the art.

A “panel” refers to a group of two or more distinct molecular species that have shown to be indicative of or otherwise correlated with a particular disease or health condition. Such “molecular species” may be referred to as “biomarkers”, with the term “biomarker” being understood to mean a biological molecule the presence or absence of which serves as an indicator of a particular biological state, for example, the occurrence (or likelihood of the occurrence) of dry eye disease in a subject. In other words, a biomarker is a characteristic that can objectively measured and evaluated as an indicator of normal biologic processes, pathogenic processes, or pharmacologic responses to a therapeutic intervention. In the context of the invention an “assay panel” or “array panel” refers to an article, typically a solid phase substrate, having a panel of capture reagents associated therewith (typically by immobilization), wherein at least one of the capture reagents is specifically reactive with a biomarker associated with dry eye disease. In some embodiments, an assay panel includes 2, 3, 4, 5, 6, 7, 8, 9, 10, 11, 12, 13, 14, 15, 16, 17, 18, 19, 20, or more (e.g., 25, 30, 35, 40, 50, 75, 100, 150, 200, 250, 500, etc., including any integer, or range of integers from 1 to 500) different detection reagents, alone or combination with other detection reagents (e.g., nucleic acid-based detection reagents, etc.) associated with the presence of dry eye disease in a subject.

A “biological sample” is a sample of biological material taken from a patient or subject. Biological samples include samples taken from bodily fluids, cells, and tissues (e.g., from a biopsy) or tissue preparations (e.g., tissue sections, homogenates, etc.). A “bodily fluid” is any fluid obtained or derived from a subject suitable for use in accordance with the invention. Such fluids include tears.

A “companion diagnostic” is a diagnostic test designed to identify subgroups of patients who may or may not benefit from a particular drug, who may have adverse reactions to the drug, or who may require different dosages of the drug.

The term “drug rescue” refers to a drug or drug candidate in the context of the reevaluation of samples and/or data from discontinued clinical trials or pre-clinical development with new or improved evaluation methods.

The term “high-throughput” refers to the ability to rapidly process multiple specimens, for example, arrays or microarrays according to the invention, in an automated and/or massively parallel manner. On the other hand, the term “multiplex” refers to the concurrent performance of multiple experiments on a single device or in a single assay. For instance, a multiplex assay using an array according to the invention allows the simultaneous detection and/or measurement of a plurality of different biomarker species in a biological sample on a single device.

A “patentable” process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

The term “positive going” marker as that term is used herein refer to a marker that is determined to be elevated in subjects suffering from a disease or condition, relative to subjects not suffering from that disease or condition. The term “negative going” marker as that term is used herein refer to a marker that is determined to be reduced in subjects suffering from a disease or condition, relative to subjects not suffering from that disease or condition.

The term “sample profiling” refers to a representation of information relating to the characteristics of a biological sample, for example, tear fluid, recorded in a quantified way in order to determine patterns or signatures of biomolecules in the particular sample.

As used herein, the singular forms “a”, “an”, and “the” include plural references unless the context clearly dictates otherwise.

As used herein, the term “about” refers to approximately a +/−10% variation from the stated value. It is to be understood that such a variation is always included in any given value provided herein, whether or not it is specifically referred to.

### SUMMARY OF THE INVENTION

It is an object of the invention to provide articles, devices, kits, and methods for diagnosing or monitoring in a subject dry eye condition, a predisposition thereto, or monitoring efficacy of therapy thereof. As described herein, assessment of one or more naturally occurring biomarker species associated with dry eye disease in biological samples, particularly tear fluids, obtained from subjects can be used for diagnosis (e.g., to screen for an initial occurrence, recurrence, progression, etc.), disease stratification (that is, to identify subjects based on underlying molecular mechanisms and/or pathways), staging, monitoring (e.g., to assess whether a subject is experiencing deterioration or improvement of clinical status over time), prognosis (e.g., predicting a future medical outcome, such as improved or worsening disease, a decreased or increased morbidity risk, or responsiveness to a particular therapeutic regimen), categorizing and determination of further diagnosis and treatment regimens in subjects suffering or at risk of suffering from dry eye disease or recurrence thereof, as well as in the context of drug development.

Thus, in one aspect, the present invention concerns DED diagnostic methods. In general, these methods comprise: obtaining a sample from a subject and measuring in the sample the level of at least one biomarker, the at least one biomarker selected from the group consisting of type 2 cytokines, cytokines known to induce infiltration or proliferation of naïve or regulatory T cells, or cytokines associated with regulatory T cells, and in particular, preferably from the group consisting of lymphotoxin alpha (LT-α, TNFbeta), Interleukin(IL)-4, granulocyte-macrophage colony-stimulating factor (GM-CSF, CSF2), IL-13, IL-3, IL-10, IL-5, and IL-9 and/or any derivative, fragment, or precursor of any of the foregoing, wherein the level of the biomarker is indicative of DED, a predisposition thereto, or the efficacy of therapy thereof, and wherein the indication of DED, or a predisposition thereto in the subject, comprises an altered (i.e., decreased or increased) level of the particular biomarker in the subject relative to at least one reference value or threshold value.

Decreased goblet cell density on conjunctival epithelium and reduced mucin production are some of the well-established pathological changes in the ocular surface in DED. The present invention is based on the surprising and unexpected discovery that in the ocular surface in DED, there is the decreased level or down-regulation of cytokines associated with type 2 immunity (and Th2 helper T cells) or regulatory T cells. Some of these cytokines include lymphotoxin alpha (also known as TNFbeta), IL-4, IL-13, IL-10, IL-3, GM-CSF (also known as CSF2), IL-5, and IL-9. It is known that Lymphotoxin alpha (TNFbeta) can induce recruitment and homing of naive or regulatory T cells to local mucosal tissues, and induce production of hyaluronic acid and tissue wound healing. Genes encoding GM-CSF and type 2 immunity associated cytokines (e.g., IL-3, IL-4, IL-5, IL-13, and IL-9) are all located on the same or very close proximity in chromosome region 5q31 and their gene expression tends to be co-regulated. It has been reported that through activating tissue resident monocytes, GM-CSF could attract and shape T cells towards type 2 T cell differentiation through upregulation of IL-4, IL-10, and IL-13. It is well known that a type 2 immunity environment favors tissue repair and wound healing and regulation of inflammation. In mucosal tissues, IL-13 is important for induction of mucin production, and it is also known that IL-13 is important for the induction of goblet cell proliferation and mucin production in conjunctival epithelium. IL-10 is a critical immune regulatory cytokine and is important for the induction/expansion of regulatory T cells. Regulatory T cells have an indispensable role in regulating adaptive immunity to limit excessive inflammation and prevent tissue damage caused by inflammation, thus maintain ocular immune tolerance in a healthy normal eye. Thus, taken together, a decreased level or down-regulated level of cytokines associated with type 2 immunity (and Th2 helper T cells) or regulatory T cells in clinical samples including tear fluid or ocular samples such as conjunctival impression cytology samples, is indicative of DED, or a predisposition thereto.

In another aspect, the present invention provides devices and methods of monitoring dry eye disease in a subject, the methods comprising: obtaining a sample from the subject; measuring in the sample the level of lymphotoxin alpha (TNFbeta), IL-4, GM-CSF (CSF2), IL-13, IL-3, IL-10, IL-5 and IL-9, and/or any of their derivatives, fragments, or precursors; and comparing the level with at least one threshold value to determine if the measured level of the particular biomarker(s) is indicative of DED, thus providing for DED monitoring.

In another aspect, the present invention provides devices and methods of monitoring efficacy of a treatment for dry eye in a subject, the methods comprising: obtaining a sample from the subject; measuring in the sample the level of lymphotoxin alpha (TNFbeta), IL-4, GM-CSF (CSF2), IL-13, IL-3, IL-10, IL-5, and IL-9, and/or any of their derivatives, fragments, or precursors; and comparing the level with at least one threshold value to determine if the measured level of the particular biomarker(s) is indicative of DED, thus providing for monitoring efficacy of a treatment for DED in the subject.

In another aspect, the present invention provides devices and methods of predicting the risk of cornea allograft rejection in a subject, the methods comprising: obtaining a sample from the subject; measuring in the sample the level of lymphotoxin alpha (TNFbeta), IL-4, GM-CSF (CSF2), IL-13, IL-3, IL-10, and/or any of their derivatives, fragments, or precursors; and comparing the level with at least one threshold value to determine if the measured level of the particular biomarker(s) is indicative of DED, thus providing methods for predicting the risk of cornea allograft rejection in the subject.

In the above aspects, the at least one threshold value maybe determined from a statistically significant number of normal control subjects not exhibiting any dry eye signs or experiencing dry eye symptoms. The sample may be fluid, such as tear fluid. The level of the biomarker may be measured by the amount or concentration of the at least one biomarker. The measuring may be by antibody-based immunoassay.

In another aspect, the present invention provides a panel of biomarkers for diagnosing dry eye, the panel comprising lymphotoxin alpha (TNFbeta), IL-4, GM-CSF (CSF2), IL-13, IL-3, IL-5, IL-10, and IL-9, and/or derivatives, fragments, or precursors thereof with at least one threshold value. The panel may provide a more detailed profile of the condition, yielding both diagnostic as well prognostic information, as compared to the results from only one or a few of the other biomarkers.

In various related aspects, the present invention also relates to devices and kits for performing the methods described herein. Suitable kits comprise at least one detection reagent species capable of binding or reacting to at least one biomarker, which biomarker(s) is(are) preferably selected from the group consisting of lymphotoxin alpha (LT-α, TNFbeta), IL-4, GM-CSF (CSF2), IL-3, IL-10, IL-13, IL-5, IL-9, and a derivative, fragment, or precursor of any of the foregoing, and instructions for using the detection reagent species to analyze a sample obtained from a subject to determine if the sample contains a reduced (or increased) level of the biomarker(s) below (or above) the threshold value that is indicative of dry eye disease. Detection reagent species preferably comprise an antibody or antigen-binding antibody fragment. While monoclonal antibodies are preferred, polyclonal antibodies can also be utilized. One or more detection reagent species in the kit may be immobilized on one or more solid substrates. The diagnostic kits may also be measured (e.g., photometrically, fluorescently, radioactively, etc.) with a suitable reader or visualized by eye. Qualitative, semi-quantitative, and quantitative analytical methods can be employed. The diagnostic kits may be rapid in vitro diagnostic tests. Thus, suitable kits comprise reagents sufficient for performing an assay according to the invention, together with instructions and algorithms for performing the described concentration calculation, correlation analysis and/or threshold comparisons.

In some embodiments, two or more different detection reagent species may be employed, in which event each detection reagent species preferably binds a different biomarker species. In some embodiments, however, two or more detection reagent species may target the same or different epitopes on the same biomarker. The diagnostic kits may further comprise information pertaining to the use of the kits.

In general, instructions include contacting a clinical sample obtained from a subject suspected of having or known to have dry eye disease with a detection reagent that binds or react with a biomarker associated with dry eye disease. The detection reagent is then used to determine if the biomarker associated with dry eye disease is present in the sample an amount indicative of dry eye disease.

Features and advantages of the invention will be apparent from the following drawings, detailed description, and appended claims.

### BRIEF DESCRIPTION OF THE DRAWINGS

A brief summary of each of the figures and tables described in this specification are provided below. This application contains at least one figure executed in color. Copies of this application with color drawings will be provided upon request and payment of the necessary fee.

FIG. 1 is a multivariate analysis of tear markers in DED patients. 2-way unsupervised hierarchical clustering of tear protein markers and patients. Column: patients, Row: tear protein markers.

FIG. 2 is a principal component analysis (PCA). A: PCA of dry eye patients using tear protein markers. B: PCA of both control subjects and dry eye patients. PCA plot: green=G1, blue=G2, red=G3, orange=G4, grey=Control.

FIG. 3 is a scatter plot comparing levels of Lymphotoxin alpha (TNFbeta) in tear fluid collected from normal control subjects and dry eye patients.

FIG. 4 is a scatter plot comparing levels of IL-4 in tear fluid collected from normal control subjects and dry eye patients.

FIG. 5 is a scatter plot comparing levels of IL-10 in tear fluid collected from normal control subjects and dry eye patients.

FIG. 6 is a scatter plot comparing levels of GM-CSF (CSF2) in tear fluid collected from normal control subjects and dry eye patients.

FIG. 7 is a scatter plot comparing levels of IL-3 in tear fluid collected from normal control subjects and dry eye patients.

FIG. 8 is an ROC curve when using Lymphotoxin alpha (TNFbeta) as a dry eye biomarker. The accuracy (area under the ROC curve) is 89.4%.

FIG. 9 is an ROC curve when using IL-4 as a dry eye biomarker. The accuracy (area under the ROC curve) is 99.0%.

FIG. 10 is an ROC curve when using IL-10 as a dry eye biomarker. The accuracy (area under the ROC curve) is 98.4%.

FIG. 11 is an ROC curve when using GM-CSF (CSF2) as a dry eye biomarker. The accuracy (area under the ROC curve) is 98.5%.

FIG. 12 is an ROC curve when using IL-3 as a dry eye biomarker. The accuracy (area under the ROC curve) is 98.3%.

FIG. 13 is a diagram of a representative lateral flow strip diagnostic device according to the invention. The lateral flow strip includes a conjugate pad with a sample receiving area, a wicking membrane with one or more test lines and a control line of corresponding antibodies.

Table 1 is a comparison between dry eye patient group and normal control group: group geometric mean, median, range, P value from T-test, and AUC (area under the curve) of ROC curve. Biomarker concentration values (pg/mL) are log 10 transformed.

### DETAILED DESCRIPTION

As those in the art will appreciate, the following detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

More specifically, the present invention relates to articles, devices, kits, and methods for diagnosis, differential diagnosis, disease stratification, monitoring, classifying, and determination of treatment regimens in subjects suffering or at risk of suffering from dry eye disease through measurement of one or more biomarkers associated with the disease.

### Biomarker Changes In Disease

The cellular changes that mark the transition from a healthy to a diseased state are frequently, if not always, mediated by changes in the level or type of constituent biomarkers, including proteins, nucleic acids, carbohydrates, and lipids. These changes can result from several different mechanisms, including changes in the abundance or expression level of certain proteins, the rate of transcription of DNA to mRNA or the translation of mRNA to protein, mRNA stability, the rate of protein turnover, or other metabolic processes. One, some, or all of these and other mechanisms may be modulated, with the result being that the synthesis and/or stability of one or more biomarker species is increased or decreased in a manner that can be detected in an assay of a biological sample. With particular regard to proteins, there may also be changes in the primary sequence of a protein conferred by alterations in the corresponding gene sequences, due to single nucleotide polymorphisms (SNPs), alternate mRNA splicing, genomic rearrangements, or any of several other mechanisms for genetic variation. There may also be changes in the processing and post-translational modification of proteins. For example, a protein may be differentially glycosylated such that alternative glycoforms can be detected.

### Analyte Detection

The presence and/or amount of a target analyte, e.g., a biomarker associated with dry eye disease, can be detected or measured in biological samples, particularly tears, obtained from subjects by any suitable method, including obtaining a small tear volume directly from a subject's eye, as well as via biopsy, swab, washing, or other technique useful to collect a biological fluid or cell sample from a patient. Particularly preferred biological samples are tear samples, as tear fluid is usually a readily accessible solution that can be obtained by relatively non-invasive sampling techniques.

Biomarkers are generally detected using biomarker-reactive reagent species immobilized on a substrate such as a solid support. A biomarker detection reagent species is one specifically reactive with an epitope of a biomarker now known or later discovered to be associated with dry eye disease. Thus, a detection reagent species refers to a reagent that is specifically reactive with a particular epitope of a biomarker antigen. Preferred detection reagent species comprise polyclonal, and even more preferably, monoclonal antibodies, or the antigen-binding fragments of such antibodies. A detection reagent may also include one or more other moieties, for example, a detectable label.

In this invention, one or more detection reagent species are immobilized on a suitable substrate, for example, plastic beads, on the surface of the detection zone of a lateral flow device, etc. In this way, the detection reagent(s) can be brought into contact with a small biological sample (e.g., from about 1 nanoliter (nL) to about 5 microliters (uL) of tear fluid) to determine if it contains one or more biomarkers associated with dry eye disease or a related disorder, for example, Sjogren Syndrome. If the sample contains the biomarker(s) of interest, the detection reagent species binds thereto to form a complex between the detection reagent species and the biomarker (or analyte) targeted by that particular species of detection reagent.

A biomarker detection array (or other configuration of multiple detection reagent species immobilized on one or more substrates) of the invention can also include other moieties reactive with biomolecules in a biological sample. For example, detection reagents reactive with disease-associated metabolites, proteins, and/or nucleic acids that encode them, can also be included. Detection reagents for these and/or other disease-associated biomarkers can also be included in a panel or on an array according to the invention.

In preferred embodiments, the arrays of the invention comprise at least two detection reagent species, each of which corresponds to (i.e., is directed against or targets for binding) a specific biomarker.

As those in the art will appreciate, immunoassay formats are particularly preferred for implementing the instant invention. Immunoassays can provide qualitative, semi-quantitative, or quantitative output. Immunoassays are biochemical tests that measure the presence and/or level of one or more substances, i.e., analytes (e.g., biomarkers such as proteins, nucleic acids, etc.), in a biological sample, for example, a small volume of tear fluid, using the reaction of an antibody or antibodies to its antigen. The assay takes advantage of the specific binding of an antibody to its antigen to form an antibody-antigen complex, a representative example of a detection reagent-biomarker complex. Antigens or antibodies can be detected or measured. In the context of the invention it is generally biomarker species that are detected.

Numerous immunoassay formats are known to those of skill in the art, who understand that the signals obtained from an immunoassay are a direct result of complexes formed between one or more antibodies (a preferred detection reagent component) and polypeptides (a representative class of biomarker) containing the necessary epitope(s) to which the antibodies bind. As used herein, the term “relating a signal to the presence or amount” of an analyte reflects this understanding. As already described, assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte.

In general, immunoassays involve contacting a sample containing or suspected of containing a biomarker of interest with at least one antibody (or antigen-binding antibody fragment or other reagent, e.g., a receptor or receptor fragment that binds the biomarker, an aptamer, etc.) that specifically binds to the biomarker. A signal is then generated indicative of the presence or amount of complexes formed by the binding of biomarkers in the sample to the antibody (or other class of detection reagent). The signal is then related to the presence or amount of the biomarker in the sample. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed., Elsevier 2005, each of which is hereby incorporated by reference in its entirety, including all tables, figures, and claims.

In preferred embodiments, the assay devices and methods known in the art can utilize labeled molecules in various sandwich, competitive, or non-competitive immunoassay formats to generate a signal that is related to the presence or amount of the biomarker of interest. Other suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation, including but not limited to, Beckman ACCESS®, Abbott AXSYM®, Roche ELECSYS®, Dade Behring STRATUS® systems, are among the immunoassay analyzers that are capable of performing immunoassays. But any suitable immunoassay may be utilized, for example, enzyme-linked immunoassays (ELISA), radioimmunoassays (RIAs), competitive binding assays, and the like.

Antibodies or other polypeptides (or other types of detection reagents, e.g., aptamers) may be immobilized onto a variety of solid supports for use in assays. Solid phases that may be used to immobilize specific binding members include those developed and/or used as solid phases in solid phase binding assays. Examples of suitable solid phases include membrane filters, cellulose-based papers, beads (including polymeric, latex, and paramagnetic particles), glass, silicon wafers, microparticles, nanoparticles, TentaGels, AgroGels, PEGA gels, SPOCC gels, and multiple-well plates. Antibodies or other detection reagents may be bound to specific zones of assay devices either by conjugating directly to an assay device surface, or by indirect binding. In an example of the later case, antibodies or other polypeptides may be immobilized on particles or other solid supports, and that solid support immobilized to the device surface.

Biological assays require methods for detection, and one of the most common methods for quantitation of results is to conjugate a detectable label to a protein or nucleic acid (or other class of detection reagent) that has affinity for one of the components (e.g., a biomarker of interest) in the biological system being studied. Detectable labels may include molecules that are themselves detectable (e.g., fluorescent moieties, electrochemical labels, ecl (electrochemical luminescence) labels, metal chelates, colloidal metal particles, radioactive labels, etc.), as well as molecules that may be indirectly detected by production of a detectable reaction product (e.g., enzymes such as horseradish peroxidase, alkaline phosphatase, etc.) or through the use of a specific binding molecule which itself may be detectable (e.g., a labeled antibody that binds to the second antibody, biotin, digoxigenin, maltose, oligohistidine, 2,4-dintrobenzene, phenylarsenate, ssDNA, dsDNA, etc.). Labels that can be directly or indirectly detected may be referred to as “signal development elements”.

Generation of a signal from the signal development element can be performed using various optical, acoustical, and electrochemical methods well known in the art. Examples of detection modes include fluorescence, radiochemical detection, reflectance, absorbance, amperometry, conductance, impedance, interferometry, ellipsometry, etc. In certain of these methods, the solid phase antibody is coupled to a transducer (e.g., a diffraction grating, electrochemical sensor, etc.) for generation of a signal, while in others, a signal is generated by a transducer that is spatially separate from the solid phase antibody (e.g., a fluorometer that employs an excitation light source and an optical detector). This list is not meant to be limiting. Antibody-based biosensors may also be employed to determine the presence or amount of analytes that optionally eliminate the need for a labeled molecule.

Preparation of solid phases and detectable label conjugates (i.e., a molecule that contains a detectable label conjugated to a detection reagent species) often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available

To obtain quantitative or semi-quantitative results, results must be compared to standards of a known concentration. This is usually done though the use of one or more standard curves. The position of the curve at response of the unknown is then examined, and so the quantity of the unknown found.

Detecting the quantity of a particular protein or other biomarker species can be achieved by a variety of methods, any of which can be readily adapted for practice of the invention. ELISA is a commonly used technique for detecting antibody or antigen levels. One of the most common methods is to label either the antigen or antibody with an enzyme, radioisotope, or fluorescence. Other suitable techniques include agglutination, flow cytometry, Luminex assays, cytometric bead arrays, and lateral flow, among others now know or later developed.

Immunoassays can involve “sandwich” approaches in which the analyte to be detected (e.g., a protein found in tears that is associated with dry eye disease) is bound by two other entities, for example, by a capture reagent immobilized on a substrate and specific for the target biomarker species and a labeled detection reagent that binds to another epitope on the targeted biomarker species. In this way the “sandwich” can be used to measure the amount of the biomarker bound between the capture and detection reagents. Sandwich assays are especially valuable to detect analytes present at low concentrations or in complex solutions (e.g., tears) containing high concentrations of other molecular species. As is known, in these sorts of assays a “capture” reagent is immobilized on a solid phase (i.e., on a substrate) such as a glass slide, plastic strip, or microparticle. A liquid biological sample (e.g., a tear sample) known or suspected to contain the targeted biomarker is then added and allowed to complex with the immobilized capture reagent. Unbound products are removed and the detection reagent is then added and allowed to bind to biomarker species that has been “captured” on the substrate by the capture reagent, thus completing the “sandwich”. These interactions can then be used to quantitate the amount of the captured biomarker species present in the biological sample.

As will be appreciated, a plurality of different dry eye disease-associated capture reagent species (e.g., 2, 5, 10, 25, 50, 100, or more capture reagent species) can be immobilized on the substrate (or on different substrates, for example, different distinguishable microparticles) in order to detect, via “capture”, a plurality of different biomarker species in a single multiplex assay. To allow simultaneous detection of multiple biomarker species in a single assay, a multiplex assay format can be used. Multiplex formats provide an array of different moieties that allow simultaneous detection of multiple analytes (e.g., different biomarker species) at multiple array addresses on a single substrate. Alternatively, when a panel of the invention is spread across multiple substrates, for example, in embodiments where different dry eye disease-associated capture or detection reagent species are immobilized on substrates that can be distinguished (e.g., differentially labeled microparticles configured for use in Luminex assays), multiple array addresses can still be readily distinguished.

Thus, in certain embodiments, the assay methods of the invention utilize immunoassays. In certain embodiments, reagents for performing such assays are provided in an assay device, and such assay devices may be included in such a kit. Preferred reagents can comprise two or more independently selected solid phase detection reagents, each of which comprises an antigen reagent species specific for its target biomarker, immobilized on the same or different substrate (here, any suitable solid support). In the case of sandwich immunoassays, such reagents can also include one or more detectably labeled antibodies, the detectably labeled antibody comprising antibody that detects the intended biomarker target(s) bound to a detectable label. Additional optional elements that may be provided as part of an assay device are described hereinafter. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994.

Certain aspects of the present invention concern diagnostic kits. Such kits comprise biomarker detection panels according the invention in order to allow performance of the methods of the invention. Such kits can also include devices and instructions for performing one or more of the methods described herein. The instructions can be in the form of labeling, which refers to any written or recorded material that is attached to, or otherwise accompanies a kit at any time during its manufacture, transport, sale, or use. For example, the term labeling encompasses advertising leaflets and brochures, packaging materials, instructions, computer storage media, as well as writing imprinted directly on kits.

In preferred embodiments, a panel of the invention will also include controls, preferably at least one positive and one negative control. Any suitable set of controls can be selected.

Additional clinical indicia may be combined with the biomarker assay result(s) of the present invention. These include other biomarkers associated or correlated with dry eye disease. Other clinical indicia which may also be combined with the assay result(s) of the present invention includes patient demographic information (e.g., weight, sex, age, race, smoking status), medical history (e.g., family history, type of surgery, pre-existing or previous diseases), and genetic information. Combining assay results/clinical indicia in this manner can comprise the use of multivariate logistical regression, loglinear modeling, neural network analysis, n-of-m analysis, decision tree analysis, etc. This list is not meant to be limiting.

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the results of an assay, most preferably an immunoassay, of the present invention, optionally together with other clinical characteristics, to arrive at a diagnosis (that is, the occurrence or nonoccurrence) of dry eye disease for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

Similarly, a prognostic risk signals a probability (“a likelihood”) that a given course or outcome will occur. A level or a change in level of a prognostic indicator, which in turn is associated with an increased probability of morbidity (e.g., worsening of the particular disease or condition) is referred to as being “indicative of an increased likelihood” of an adverse outcome in a subject.

In preferred diagnostic embodiments, the methods of the invention allow for diagnosing the occurrence or nonoccurrence of a disease, particularly dry eye disease, and the assay result(s) is/are correlated to the occurrence or nonoccurrence of the particular disease. For example, each of the measured biomarker levels (e.g., as concentration(s)) may be compared to a threshold value, which may be different for each biomarker species (or other analyte or biomarker to be studied in a given assay). The terms “correlating”, “correlated with”, and “associated with” as used herein in reference to the use of biomarkers refers to comparing the presence or amount of the biomarker(s) in a patient to its presence or amount in persons known to suffer from, or known to be at risk of, a given condition; or in persons known to be free of a given condition. Often, this takes the form of comparing an assay result in the form of a biomarker concentration to a predetermined threshold selected to be indicative of the occurrence or nonoccurrence of a disease or the likelihood of some future outcome.

In this context, “diseased” is meant to refer to a population having one characteristic (the presence of a disease or condition or the occurrence of some outcome) and “non-diseased” is meant to refer to a population lacking the characteristic. While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific therapy that is highly efficacious and has a low level of risk, few tests are needed because clinicians and patients are willing to accept substantial diagnostic uncertainty. On the other hand, in situations where treatment options are less effective and more risky, clinicians and patients often require a higher degree of diagnostic certainty before adopting a particular treatment regimen. Thus, cost/benefit analysis is involved in selecting a diagnostic threshold.

A variety of methods may be used by to arrive at a desired threshold value for use in these methods. For example, the threshold value may be determined from a population of normal subjects by selecting a concentration representing the 75^{th}, 85^{th}, 90^{th}, 95^{th}, or 99^{th} percentile of the biomarker measured in such normal subjects. Alternatively, the threshold value may be determined from a “diseased” population of subjects, e.g., those suffering from a disease such as a dry eye disease or having a predisposition for dry eye disease, its recurrence, or progression, by selecting a concentration representing the 75^{th}, 85^{th}, 90^{th}, 95^{th}, or 99^{th} percentile of the biomarker measured in such subjects. In another alternative, the threshold value may be determined from a prior measurement of the biomarker in the same subject, where a prior “baseline” result is used to monitor for temporal changes in a biomarker level; that is, a temporal change in the level of the biomarker in the subject may be used for diagnostic and/or prognostic purposes.

The foregoing discussion is not meant to imply, however, that the levels of biomarkers measured in assays of the invention must be compared to corresponding individual thresholds. Methods for combining assay results can comprise the use of multivariate logistical regression, loglinear modeling, neural network analysis, n-of-m analysis, decision tree analysis, calculating ratios of markers, etc. This list is not meant to be limiting. In these methods, a composite result that is determined by combining individual biomarker data or results may be treated as if it is itself a marker; that is, a threshold may be determined for the composite result as described herein for individual biomarkers, and the composite result for an individual patient compared to this threshold.

Population studies may also be used to select a decision threshold. The Receiver Operating Characteristic (“ROC”) arose from the field of signal detection theory developed during World War II for the analysis of radar images, and ROC analysis is often used to select a threshold able to best distinguish a “diseased” subpopulation from a “non-diseased” subpopulation. A false positive in this case occurs when a subject tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1—specificity, the ROC graph is sometimes called the sensitivity versus (1—specificity) plot. A perfect test will have an area under the ROC curve of 1.0; a random test will have an area of 0.5. A threshold is selected to provide an acceptable level of specificity and sensitivity.

Thus, the ability of a particular test to distinguish two populations can be established using ROC analysis. For example, ROC curves established from a “first” subpopulation which is predisposed to future disease or disease-related changes, and a “second” subpopulation which is not so predisposed can be used to calculate a ROC curve, and the area under the curve provides a measure of the quality of the test. Preferably, the tests described herein provide a ROC curve area greater than 0.5, preferably at least 0.6, more preferably 0.7, still more preferably at least 0.8, even more preferably at least 0.9, and most preferably at least 0.95.

In certain aspects, the measured concentration of one or more target biomarkers (e.g., disease-associated serum autoantibodies), or a composite of results, may be treated as continuous variables. For example, any particular concentration can be converted into a corresponding probability of some outcome for the subject. In yet another alternative, a threshold that can provide an acceptable level of specificity and sensitivity in separating a population of subjects into “bins” such as a “first” subpopulation (e.g., which is predisposed to one or more future changes in disease status, the occurrence or recurrence of disease, a disease classification or stratification, etc.) and a “second” subpopulation which is not so predisposed.

As discussed above, suitable tests may exhibit one or more of the following results on these various measures: a specificity of greater than 0.5, preferably at least 0.6, more preferably at least 0.7, still more preferably at least 0.8, even more preferably at least 0.9 and most preferably at least 0.95, with a corresponding sensitivity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; a sensitivity of greater than 0.5, preferably at least 0.6, more preferably at least 0.7, still more preferably at least 0.8, even more preferably at least 0.9 and most preferably at least 0.95, with a corresponding specificity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; at least 75% sensitivity, combined with at least 75% specificity; a ROC curve area of greater than 0.5, preferably at least 0.6, more preferably 0.7, still more preferably at least 0.8, even more preferably at least 0.9, and most preferably at least 0.95; an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1—specificity)) of greater than 1, at least 2, more preferably at least 3, still more preferably at least 5, and most preferably at least 10; and or a negative likelihood ratio (calculated as (1—sensitivity)/specificity) of less than 1, less than or equal to 0.5, more preferably less than or equal to 0.3, and most preferably less than or equal to 0.1.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

Measures of test accuracy may be obtained as described in Fischer, et al., Intensive Care Med. 29: 1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

### Antibodies

Antibodies (or antigen-binding antibody fragments and the like) used in the immunoassays described herein preferably specifically bind to a biomarker of the present invention associated or correlated with DED. The term “specifically binds” is not intended to indicate that an antibody binds exclusively to its intended target since an antibody is capable of binding to any molecule displaying the epitope(s) to which the antibody binds. Rather, an antibody “specifically binds” if its affinity for its intended target is about 5-fold greater when compared to its affinity for a non-target molecule which does not display the appropriate epitope(s). Preferably the affinity of the antibody will be at least about 5-fold, preferably 10-fold, more preferably 25-fold, even more preferably 50-fold, and most preferably 100-fold or more, greater for a target molecule than its affinity for a non-target molecule. In preferred embodiments, preferred antibodies bind with affinities of at least about 10⁶ M⁻¹ or 10⁷ M⁻¹ to about 10¹² M⁻¹ and preferably between about 10⁸ M⁻¹ to about 10⁹ M⁻¹, about 10⁹ M⁻¹ to about 10¹⁰ M⁻¹, or about 10¹⁰ M⁻¹ to about 10¹² M⁻¹.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art.

Numerous publications discuss the use of phage display technology to produce and screen libraries of polypeptides for binding to a selected analyte. See, e.g., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome that encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target analyte can then be synthesized in bulk by conventional means. See, e.g., U.S. Pat. No. 6,057,098.

The antibodies that are generated by these methods may then be selected by first screening for affinity and specificity with the purified biomarker of interest and, if required, comparing the results to the affinity and specificity of the antibodies with biomarkers that are desired to be excluded from binding. The screening procedure can involve immobilization of the purified biomarkers in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein or other type of biomarker, the purified target analyte acts as a standard with which to judge the sensitivity and specificity of the immunoassay using the antibodies that have been selected. Because the binding affinity of various antibodies may differ, and since certain antibody pairs (e.g., in sandwich assays) may interfere with one another sterically, etc., assay performance of an antibody may be a more important measure than absolute affinity and specificity of an antibody.

### Applications

The detection reagents, panels, arrays, and kits of the invention have numerous applications, including to monitor, prognose, diagnose, or in conjunction with treatment of a subject or patient having, dry eye disease.

The arrays of the invention can be used to assess biological samples from patients known to have, suspected of having, or to have been previously diagnosed and/or treated for having, a particular disease, for example, a dry eye disease such as Sjogren's Syndrome, as well as to screen subjects not previously known or suspected to have a particular disease. At the time of screening, the subject or patient may be symptomatic or asymptomatic. Biomarker levels corresponding to some or all of the biomarker-reactive reagent species, or antigens, disposed on the array can be used prognostically, for example, to determine if a patient's disease is amenable to a particular treatment, to monitor disease progression and/or effectiveness of a therapeutic regimen, to assess disease aggressiveness of disease, and/or to identify likelihood of recurrence. The arrays of the invention can also be employed for diagnostic and screening purposes. For example, arrays can be configured to use in diagnosing one or more subtypes of dry eye disease.

The devices and arrays of the invention can also be used as a companion diagnostic, for example, to identify patients as likely responders or non-responders to a particular drug treatment or other therapeutic regimen, as well as for assessing the stage of a patient's disease as biomarker profiles are likely to change during disease progression. For example, tumors express different proteins (and thus produce different antigens) to meet the different requirements at each phase of development. Similarly, autoimmune diseases can “flare” at different times.

Data sets from diseased samples can also be correlated with clinical data. Antibody profiles can be used to predict disease severity or clinical outcome, which will be useful for prognostic applications. The use of biomarker panels will allow different stages of disease to be assessed, as the biomarker profile of a given sample will allow the particular stage of a given disease to be discerned, thereby allow the most effective therapeutic intervention(s) to be employed.

The devices and arrays of the invention will also find use in drug development, both in the discovery and clinical development phases, particularly for biologic drugs such as antibodies and other recombinant proteins as well as cell- or vesicle-based drug delivery systems. Drugs of this class can, at least in some cases, elicit immune responses that can be advantageous (e.g., positive response to a vaccine) or harmful (e.g., severe adverse autoimmune reaction). Similarly, immune responses can also result from the administration of small molecule drugs, as a result of changes to cells and tissues following administration of the drug. The ability to monitor immune responses to biologic and small molecule drugs in clinical trials has never been more important. There is value in monitoring not only cellular immune responses but also humoral immune responses, and comparison of serum antibody profiles before and after treatment can help predict a favorable drug response. Positive responders to a drug will exhibit a different baseline humoral immune status to their disease. This is especially valuable in the case of immunomodulator class drugs that work by modifying an existing immune response rather than stimulating one de novo. By comparing data sets from non-responders to those who respond positively or negatively to a particular drug (or drug combination), panels can be defined for analyzing different groups of autoantibodies. Such panels will allow the identification of patients likely to respond to a particular therapy. Similarly, differences between responders and non-responders in the response profiles for a particular biomarker can be used to assess whether a patient is benefiting from a particular therapeutic regimen.

As will be appreciated, different clinical study designs will allow the development of biomarker panels that address different needs within drug development and therapy. For example, identifying responders versus non-responders will allow clinicians to select responders prior to treatment through the use of a companion diagnostic test based on response-predictive biomarker panel profile. Similarly, to select patient cohorts in clinical trials, biomarker profiles predictive for a positive drug response can be used to screen subjects prior to their recruitment into a clinical trial. This will ensure that only suitable candidates are included, and it may also be useful in gaining early drug approval. Also, information on drug non-response can assist regulatory bodies during consideration of drugs for approval or during post-approval surveillance (i.e., during a Phase IV clinical trial).

Another area of drug development where the instant invention will find application is in the area of “drug rescue” by helping to define the patient population(s) amenable to successful treatment as well as those who are unlikely to respond, or perhaps even more important, those who will experience an adverse reaction if administered the drug. In other words, a retrospective analysis of patient samples from a drug candidate that failed at some point in clinical development can be used to define the biomarker panel profile(s) (or signature(s)) predictive of a positive drug response. That information can then be used to define subsequent patient cohorts for further study and treatment. This process, which may be iterated, can revitalize drugs that have fallen out of conventional clinical development due to poor or insufficient evidence of efficacy. The biomarker panel profile(s) predictive of a positive drug response can then be used to reselect likely responders, which can lead to further clinical evaluation of the previously failed drug candidate but with a much greater likelihood of ultimately achieving drug approval.

### EXAMPLES

The following Examples are provided to illustrate certain aspects of the present invention and to aid those of skill in the art in its practice. These Examples are in no way to be considered to limit the scope of the invention in any manner, and those having ordinary or greater skill in the applicable arts will readily appreciate that the specification thoroughly describes the invention and can be readily applied to carry out the objects and obtain the ends and advantages mentioned, as well as those inherent therein.

### Example 1

### In Vitro Diagnostic Test of Dry Eye: Detection of Levels of Dry Eye Biomarkers in Tear Film in Subjects of Having Dry Eye

### 1. Introduction

This example describes investigation of Lymphotxin alpha, GM-CSF, IL-4, IL-3, and IL-10 as biomarkers for diagnoses of subjects suspecting of having dry eye using an in vitro diagnostic test that measures biomarker levels in tear fluid. In this study, dry eye patients and normal control subjects were recruited based on clinical parameters and symptomatic assessments and tested for the level of biomarkers in their tear fluid and were compared.

### 2. Purpose

It is well known that standard clinical measurements of DED have large variability and poor reliability and many of the clinical procedures for DED diagnosis and monitoring are largely unrepeatable. Furthermore, clinical signs are often poorly associated with subjective, patient-reported symptoms.

Poor correlation or complete discordance between ocular symptoms and clinical signs, and between different clinical objective measures in dry eye disease has been major challenge in disease research and drug development. Identification and confirmation of biomarkers that could be used in IVD tests for the underlying molecular and cellular components contributing to the heterogeneity of ocular surface disease pathology and manifestation is of significant interest.

In this example, patients with dry eye disease and normal controls were subjected to in vitro diagnostic test measuring the level of biomarkers in the tear film and the utility of the biomarkers was evaluated for the diagnosis of dry eye in conjunction with other methods of clinical evaluation.

### 3. Methods and Materials

### Patients

The first biomarker dataset contained biomarker test results from 85 dry eye patients and 15 normal control subjects. Clinical diagnostic test results included subjective symptoms (Ocular Surface Disease Index, OSDI, a dry eye symptom assessment questionnaire), Schirmer test (without anesthesia) results, tear break-up time (TBUT) test results, corneal staining, conjunctiva staining, and other general ophthalmic examinations such as visual acuity and slit lamp examination. In this dataset, normal control subjects had an OSDI score of less than 13, TBUT equal or greater than 5 seconds, and a corneal fluorescein staining score less than 4 (NEI scale). Dry eye patients had an OSDI score of equal or greater than 23 and a TBUT shorter than 7 seconds.

The second biomarker dataset contained biomarker test results from the study eye of 33 clinically diagnosed dry eye patients. These dry eye patients had an OSDI score equal or greater than 23, a TBUT shorter than 7 seconds, and a corneal staining score at least 3 (NEI scale).

### Tear Collection

For biomarker studies, non-stimulated tear fluids (˜3 ul) were collected from the tear lake inside the lateral conjunctival sac of the inferior fornix using a glass microcapillary tube (without anesthesia).

### Biomarker Detection

Biomarker levels in tear fluids were measured with an antibody-based immunoassay for each of the biomarkers. A different antibody-based detection reagent was used for each biomarker. Lymphotoxin, IL-4, IL-3, CSF2 (GM-CSF), and IL-10 were included among the biomarkers of interest, among other protein analytes.

### Statistical Analysis

Biomarker concentration values were first log transformed. Geometric mean, median, range, and P value from T tests were then determined. Specificity, sensitivity (true positive rate, TPR), and false positive rate (FPR) were calculated. Accuracy and ROC plots were generated.

### 4. Results

### Clustering Analysis and Principle Component Analysis (PCA) of DED Patients

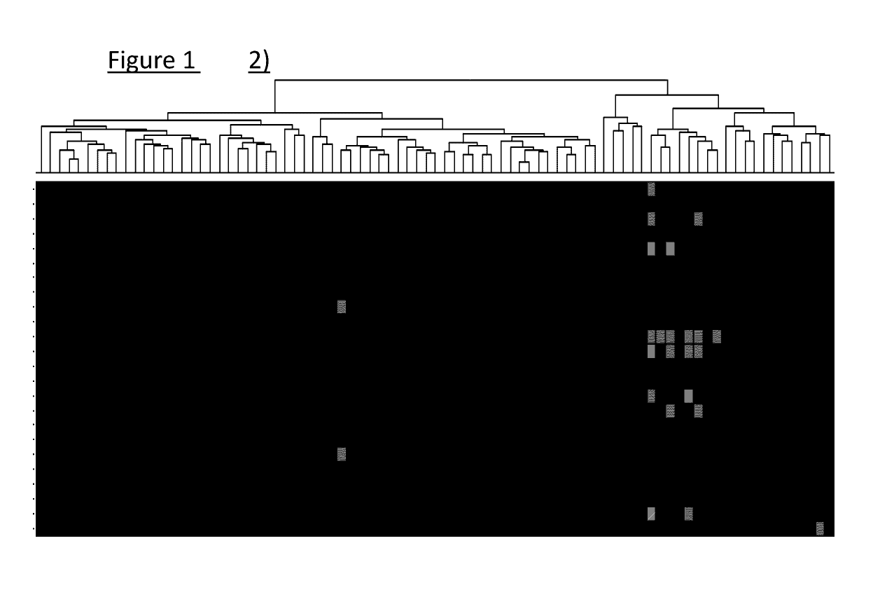

An unsupervised approach using hierarchical clustering analysis was employed to analyze DED patients based on their tear marker profiles Four (4) distinct patient subgroups were apparent within the dataset (FIG. 1). 31 DED subjects was present in Subgroup 1, 29 in Subgroup 2, and 5 and 20 patients in Subgroups 3 and 4, respectively. Similar patterns were observed when clustering analysis was conducted using data from either the study eye from each patient (prospectively defined as the worst eye based on corneal staining at the Screening visit), or taking the average of the two fellow eyes. Consistent with DED being a bilateral ocular condition, it was found previously that both clinical parameters and many tear cytokines and protein markers are comparable between fellow eyes.

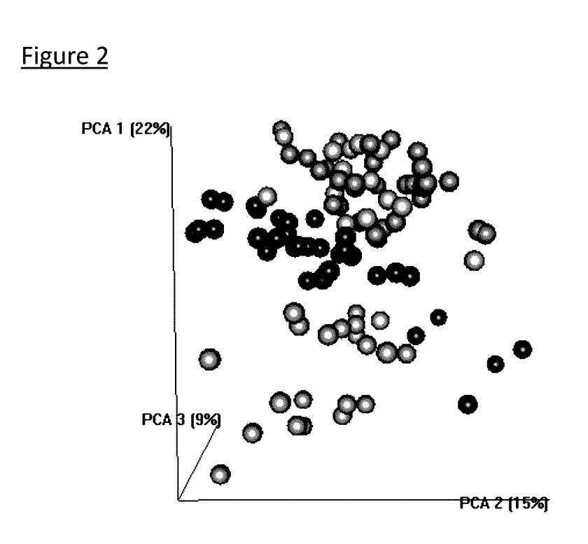

These subgroups of patients revealed by clustering analysis were also distinguishable by principal component analysis (PCA) based on their tear marker profiles (FIG. 2). Interestingly, Subgroup 1 (FIG. 2, Green color) was non-distinguishable from normal control group (FIG. 2, silver color) in PCA. Patients in Subgroup 1 were thus termed as normal-like subjects with no obvious differences in tear marker profiles from the non-DED control subject group, and they were thus excluded from the DED group in subsequent differential analysis for selecting biomarkers for DED.

Comparisons between Dry Eye Group and Normal Control Group

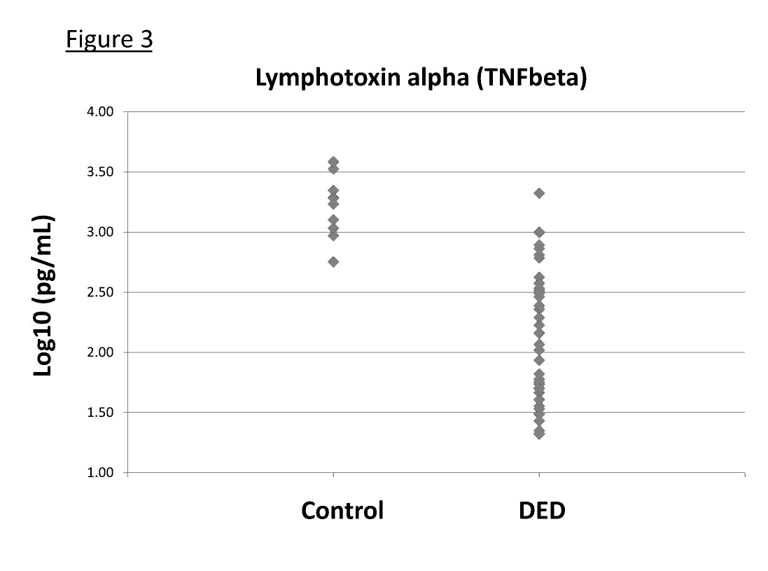

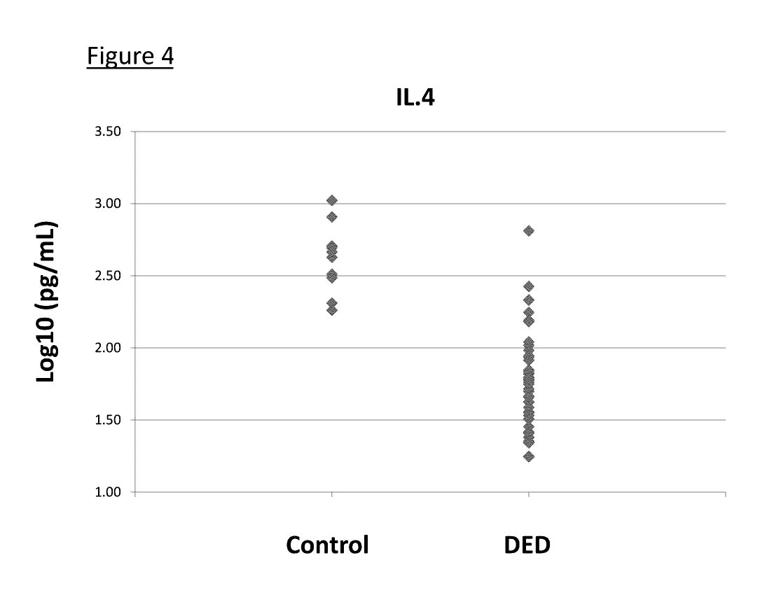

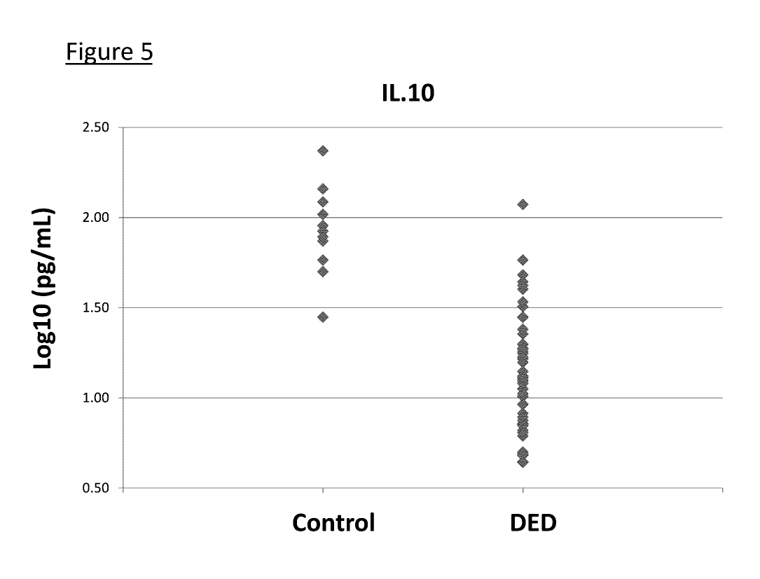

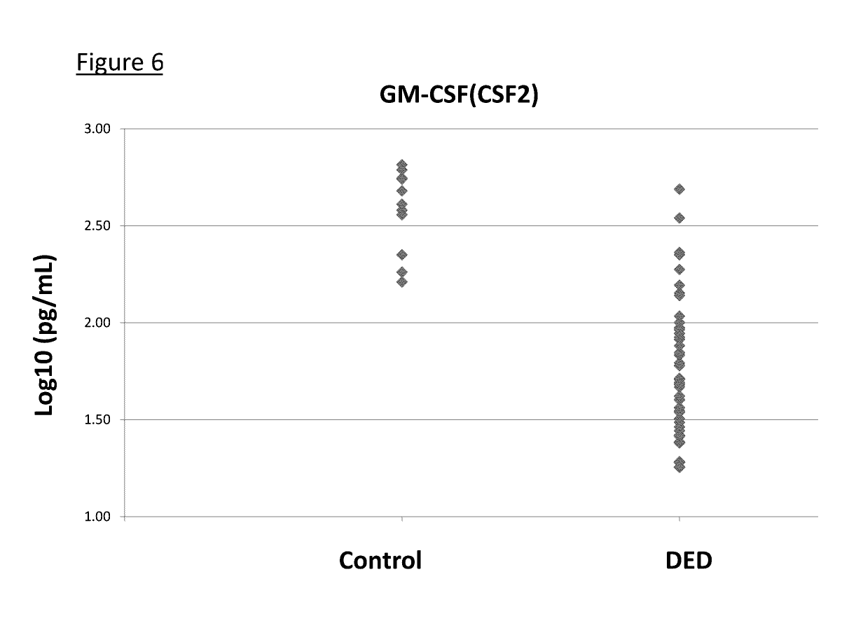

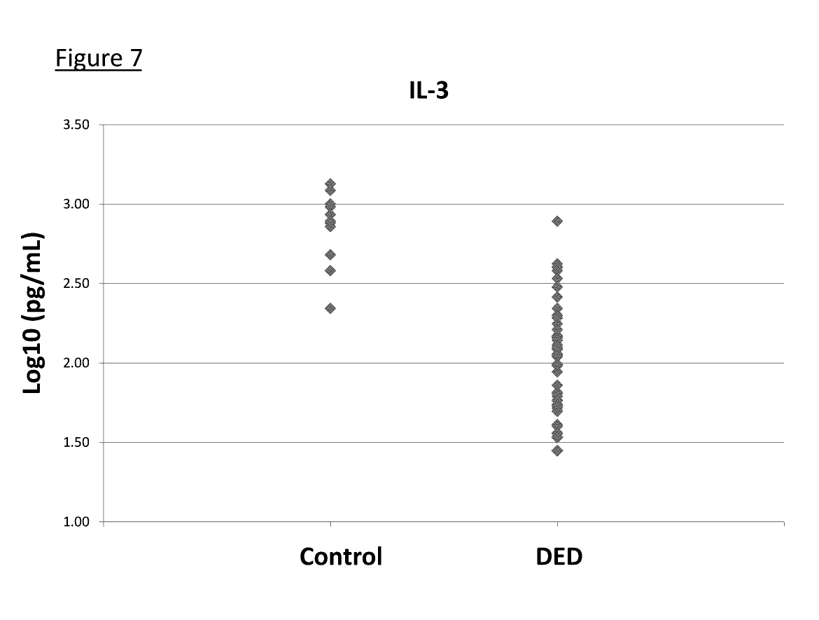

Results from biomarker tests were compared between the dry eye patient group and normal control group with T-testing. DED biomarkers were identified which were significantly down-regulated in the DED group compared with the normal control group (P<0.0001, T test), and the scatter plots of the top 5 down-regulated biomarker candidates are shown in FIG. 3-7, including Lymphotoxin alpha, IL-4, IL-3, CSF2 (GM-CSF), and IL-10. The average reduction was approximately 10 fold or higher (delta is 1 in log 10 space). The geometric mean, median, range, and P values from the T-tests of the 5 selected biomarkers are listed in Table 1.

### ROC Curve for Dry Eye Biomarkers

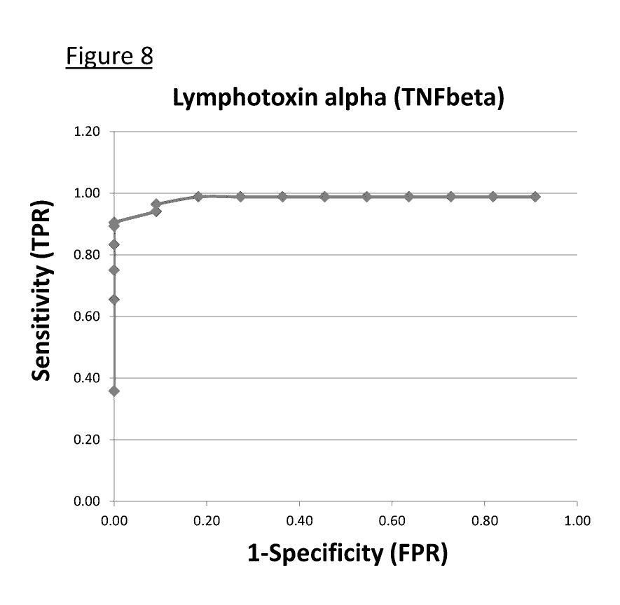

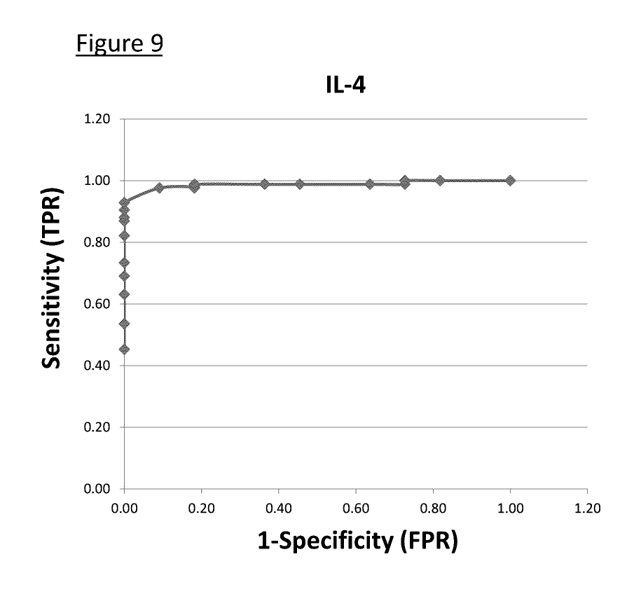

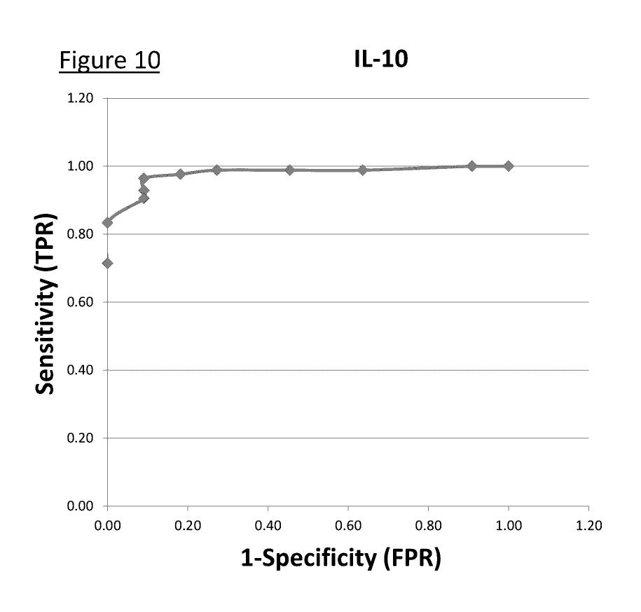

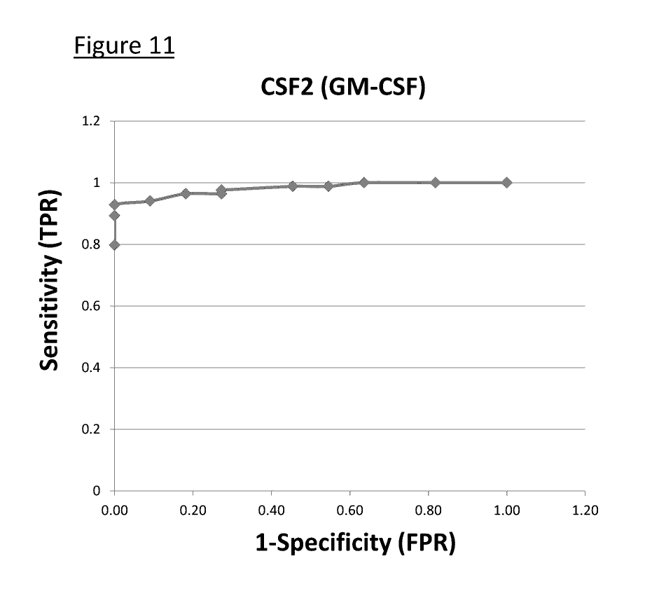

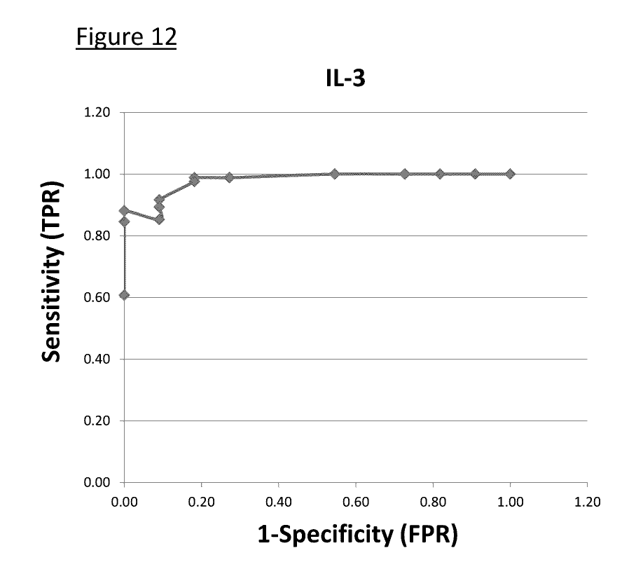

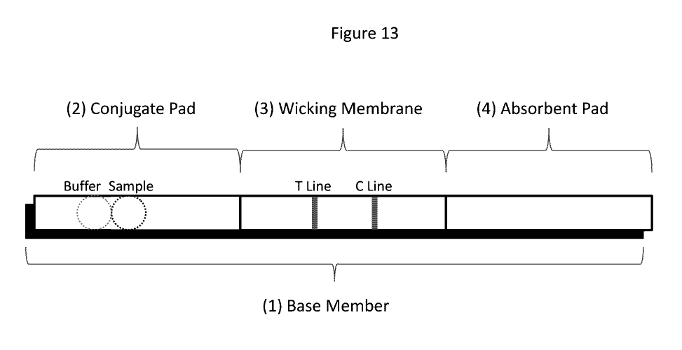

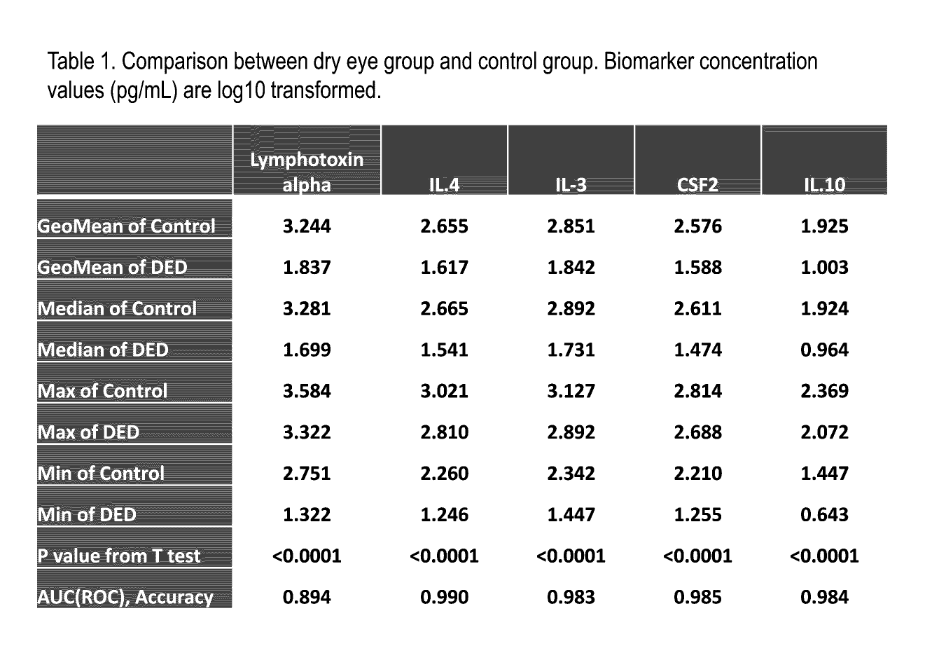

Specificity and sensitivity were calculated for each one of these 5 biomarkers as a diagnostic test for dry eye individually. ROC curves were generated using TPR and FPR for each biomarker (See FIGS. 8-12). ROC curve plots true positive rate (sensitivity) versus false positive rate (1—specificity) of various cutoff value for each biomarker level in tear fluid. The area under the ROC curve (AUC) was also calculated and this area is the accuracy. The ROC curve is useful for comparing the performance of different tests. The AUC of the ROC of accuracy were 89.4% for Lymphotoxin alpha, 99.0% for IL-4, 98.3% for IL-3, 98.5% for CSF2, and 98.4% for IL-10 (see Table 1).

### Cutoff Threshold for Dry Eye Biomarkers

If 800 pg/mL of Lymphotoxin alpha was set as the cutoff threshold for a dry eye diagnostic test with tear fluid, the specificity and sensitivity of the test would be 91% and 96%, respectively, in this dataset. The positive predictive value was 0.8% and the negative predictive value was 76.9%. If 200 pg/mL tear level of IL-4 was set as cutoff threshold, the sensitivity and specificity would be 97.6% and 90.9%, respectively. The positive predictive value was 98.8% and the negative predictive value was 83.3%. If 45 pg/mL tear level of IL-10 is set as cutoff threshold, the specificity and sensitivity was 90.9% and 96.4%, respectively, and the positive predictive value and the negative predictive value was 98.8% and 76.9%, respectively. If 300 pg/mL tear level of IL-3 was set as cutoff threshold, the specificity and sensitivity was 90.9% and 91.7%, respectively, and the positive predictive value and the negative predictive value was 98.7% and 58.8%, respectively.

### Confirmation Study of Dry Eye Biomarker Tests

In a second and separately conducted clinical study which contains clinical and biomarker data from 33 dry eye patients, biomarker tests described above were evaluated. In this confirmation study dataset, dry eye patients (the study eyes) met the clinical criteria of OSDI score equal or greater than 23, TBUT equal or shorter than 5 seconds, and corneal staining (NEI) at 3 or higher. Compared with clinical diagnosis, using a cutoff threshold value of 800 pg/mL for Lymphotoxin alpha in tear fluid predicted 28 out of 33 dry eye patients correctly (28/33, sensitivity is 84.8%); a cutoff value of 200 pg/mL for IL-4 in tear fluid predicted 31 patients correctly (31/33, sensitivity is 93.9%); a cutoff value of 45 pg/mL for IL-10 in tear fluid predicted 27 patients correctly (27/33, sensitivity is 81.8%); a cutoff value of 300 pg/mL for IL-3 in tear fluid predicted 29 patients correctly (29/33, sensitivity is 87.9%); and a cutoff value of 150 pg/mL for CSF2 in tear fluid predicted 26 patients correctly (26/33, sensitivity is 78.8%), respectively.

### Example 2

### In Vitro Diagnostic (IVD) Kit for Detection of Dry Eye Using Enzyme-Linked Immunosorbent Assay (ELISA)

An IVD test kit for dry eye using an ELISA can include at least one detection reagent species that binds at least one of the biomarkers of the present invention. Biomarkers in the tear sample can be immobilized on a solid support (usually a polystyrene 96- or 384-well microtiter plate) either non-specifically (via adsorption to the surface) or specifically (via capture by another antibody specific to the same antigen, in a “sandwich” ELISA). After the biomarker analyte is immobilized, a secondary or detection antibody that binds to the same biomarker is added, forming a complex with the antigen. The detection antibody can be covalently linked to an enzyme such as horseradish peroxidase, or can itself be detected by a secondary antibody that is linked to an enzyme through bioconjugation. A chromogenic substrate such as TMB is added and signal generated from the assay is measured with an absorbance plate reader.

### Example 3

### Multiplex Panel IVD Kit for Detection of Dry Eye

A multiplexed panel IVD test kit for dry eye can diagnose a subject of suspected of having dry eye by detecting more than one of the biomarkers of the present invention using Meso Scale Diagnostics (MSD) electrochemiluminescent detection technology, Luminex multiplex bead array assay, or Protein microarray (antibody array) technology analyzing multiple dry eye-specific biomarkers simultaneously. One example is an IVD Kit for simultaneous detection of LT-a and IL-4 in tear and diagnosis of dry eye.

### Example 4

### A Point of Care (POC) Lateral Flow Immunoassay Diagnostic Device For Rapid Detection of Dry Eye

In this example, the dry eye biomarker selected for testing is LTα, which is labeled with colored cellulose nanobeads.

### A. Preparation of a Lateral Flow Immune Assay Strips

Conjugate Pad: Anti-LTα monoclonal antibodies are conjugated with colored particles, in this example, colored cellulose nanobeads (CNB). Purified CNB particle labeled anti-LTα Mab conjugate (0.025%) is sprayed at 10 ul/cm on 18 mm fiberglass conjugate pad, then dries.

Nitrocellulose (NC) membrane: on NC membrane, a test line is dispensed with anti-LTα monoclonal antibody at 1 mg/ml. For a control line, 1 mg/ml of goat anti-mouse antibody is dispensed on the membrane downstream of the test line, dry the NC membrane.

Assemble NC membrane, conjugate pad, sample pad, wick and backing into cards. Cut cards into strips of 5 mm and assemble strips into cassettes.

### B. Tear Test

A 3 uL of tear sample is collected from a subject suspected of having dry ere and is applied to the conjugate pad in the LTα test cassette. A 50 uL of buffer is then applied to the conjugate pad upstream of where the sample is applied. On the conjugate pad, the tear sample is contacted with colored particles (CNB) that are labeled with anti-LTα antibodies. LTα present in the tear sample binds to the labeled anti-LTα antibodies. The sample then further moves to the detection region of the NC membrane comprising a test line with anti-LTα antibodies, thereby capturing the LTα that are bound by CNB-labeled anti-LTα antibodies and preventing the colored complex from moving through, thus forming a concentrated LTα labeled colored particles in the test line and resulting in a colored band. A detectable signal begins to appear in the test line after 10 minutes. This can be detected visually or with a reader device. In the absence of LTα in the sample, all of the labeled anti-LTα antibodies move past the detection zone without forming a colored band. No detectable signal indicates the subject from whom the tear sample was collected has dry eye.

Although only some embodiments of the invention have been described in detail above, those skilled in the art would readily appreciate that many modifications are possible in the embodiments without materially departing from the novel teachings and advantages of the invention. Accordingly, such modifications are intended to be included within the scope of the invention.

As one modification of the present invention, tear sample is mixed first with buffer and the mixture is then applied to the conjugate pad.

As one further modification of the present invention, the analyte of interest in the tear sample comprises a plurality of analytes to be detected, the conjugate pad is impregnated with another diffusively bound conjugate comprising a fourth binder specific and binding to another analyte and a colored particulate material (such as a different colored CNB particle), the NC membrane has further another test line disposed between the test line and the control line, and a fifth binder specific to said another analyte is fixed to said another test line. For example, the first analyte is LTα and the other analyte is IL-4.

As another modification of the present invention, the test result can be quantified with a reader device based on the intensity of the color band at test line(s).

It will be apparent to a person skilled in the art that the present invention may also be used in veterinary medicine for mammals.

While specific examples to practice the invention have been provided, it will be appreciated that various modifications and improvements may be made by a person skilled in the art without departing from the spirit and scope of the present invention.

All of the devices, methods, and compositions described and claimed herein can be made and executed without undue experimentation in light of the present disclosure. While the devices, methods, and compositions of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention claimed is:

1. A method of diagnosing or monitoring dry eye disease, or a predisposition thereto, or monitoring efficacy of therapy therefor, in a subject, the method comprising measuring in a sample taken from the subject a level of at least one biomarker selected from the group consisting of lymphotoxin alpha (TNFbeta), Interleukin(IL)-4, IL-3, IL-10, granulocyte-macrophage colony-stimulating factor (GM-CSF, CSF2), IL-13, IL-5, and IL-9, and/or any derivative, fragment, or precursor, of any of the foregoing, wherein the level of the biomarker is indicative of dry eye disease, a predisposition thereto, or the efficacy of therapy therefor, and wherein the indication of the dry eye disease, or a predisposition thereto, or the efficacy of therapy therefor, in the subject comprises a reduced level of the at least one biomarker, or any derivative, fragment, or precursor of any of the foregoing, in the subject relative to a reference value.

2. A method according to claim 1, wherein the reduced level of Lymphotoxin alpha, or any derivative, fragment, or precursor thereof, is lower than 650 pg/mL.

3. A method according to claim 1, wherein the reduced level of IL-4, or any derivative, fragment, or precursor thereof, is lower than 200 pg/mL.

4. A method according to claim 1, wherein the down-regulation of the level of IL-13 is lower than 300 pg/mL.

5. A method according to claim 1, wherein the down-regulation of the level of IL-10 is lower than 50 pg/mL.

6. A method according to claim 1, wherein the down-regulation of the level of CSF2 (GM-CSF) is lower than 150 pg/mL.

7. An in vitro diagnostic kit for diagnosing or monitoring in a subject a dry eye disease, a predisposition thereto, or monitoring efficacy of therapy therefor, comprising:
(i) a detection reagent specific for at least one of lymphotoxin alpha, IL-4, IL-13, IL-10, CSF2 (GM-CSF), or IL-9;
(ii) instructions for using the detection reagents to analyze the level of said lymphotoxin alpha, IL-4, IL-13, IL-10, CSF2(GM-CSF), or IL-9 in the biological sample obtained from a subject to determine if the level of biomarker is indicative of dry eye disease;
(iii) optionally, a reference substance for said lymphotoxin alpha, IL-4, IL-13, IL-10, CSF2(GM-CSF), or IL-9 for normalizing data; and
(iv) an information sheet for comparing the level of lymphotoxin, IL-4, IL-13, IL-10, CSF2(GM-CSF), or IL-9 to a reference level for said lymphotoxin alpha, IL-4, IL-13, IL-10, CSF2(GM-CSF), or IL-9 indicative of dry eye disease in said subject.

8. A diagnostic kit of claim 7, wherein said biological samples are tear samples.

9. A diagnostic kit according to claim 7, wherein said information sheet indicates one or more of the following:
(i) a measured level of lymphotoxin lower than 650 pg/mL is indicative of dry eye disease;
(ii) a measured level of IL-4 lower than 200 pg/mL is indicative of dry eye disease;
(iii) a measured level of IL-3 lower than 400 pg/mL is indicative of dry eye disease;
(iv) a measured level of IL-13 lower than 150 pg/mL is indicative of dry eye disease;
(v) a measured level of IL-10 lower than 50 pg/mL is indicative of dry eye disease; and/or
(vi) a measured level of CSF2(GM-CSF) lower than 150 pg/mL is indicative of dry eye disease.

10. A diagnostic kit according to claim 7, wherein the kit is used to monitor the progression or status of dry eye disease in the subject, or to monitor the efficacy of a therapy to treat dry eye disease.

11. A diagnostic kit according to claim 7, wherein the kit is used to diagnose presence or predict development of ocular surface inflammation, or to prognosis of development of cornea allograft rejection.

12. A diagnostic kit according to claim 7, wherein at least one of the detection reagent species comprises an antibody or antigen-binding antibody fragment.

13. A diagnostic kit according to claim 7, wherein the detection reagent is immobilized on a solid substrate.

14. A lateral flow immunoassay device for diagnosing or monitoring in a subject a dry eye disease, a predisposition thereto, or monitoring efficacy of therapy therefor, comprising: a base member; and a horizontal array disposed on said base member, the horizontal array comprising:
(i) a sample receiving pad being located on one end of the base member, which receive a tear sample;
(ii) a conjugate pad being distinct from the sample receiving pad, being in contact with the sample receiving pad, and comprising a diffusively bound conjugate, which forms a first immuno-complex with a dry eye biomarker of the tear in the conjugate pad, the conjugate comprising a first binder specific to the biomarker, and a label;
(iii) a wicking membrane being in contact with the conjugate pad, and having a second binder, which is immobilized in a test line of the wicking membrane, is specific to the biomarker, and which combines with the first immuno-complex to form a second immuno-complex fixed to the test line, and which receives the tear from the conjugate pad; and
(iv) the wicking membrane further comprises a third binder which does not bind to the biomarker but binds to the first binder and is immobilized in a control line of the wicking membrane, the control line being located downstream of the test line.

15. A device according to claim 14 that further comprises at least one of the following:
(i) the label is a color particle material, a colored cellulose nanobead, a gold nanoparticle, a color-changed enzyme, colored, fluorescent or paramagnetic latex particle or a fluorescent material;
(ii) at least one of the dry eye-specific markers is selected from the group consisting of LTα, IL-4, IL-13, GM-CSF, IL-3, IL-5, IL-10, IL-9, and/or any derivative, fragment, or precursor of any of the foregoing;
(iii) the first, second, and third binder are selected from a group consisting of an antibody, antigen-binding antibody fragment, a nucleic acid aptamer, and a hapten;
ivd) the horizontal array further comprises an absorbent pad disposed on the other end of the base member and being contact with the wicking membrane and having pores to absorb the tear from the wicking membrane;
(v) the conjugate pad is made of non-absorbent material of fiberglass pad, polyester, or rayon
(vi) the first binder is specific to a first epitope or a first ligand of the dry eye-specific biomarker and the second binder is specific to a second epitope or a second ligand of the biomarker.

16. A device according to claim 14, wherein the analyte of interest in the tear sample comprises a plurality of analyte, the conjugate pad is impregnated with another diffusively bound conjugate comprising a fourth binder specific and binding to another analyte and a colored particulate material, and the wicking membrane has further another test line disposed between the test line and the control line, and a fifth binder specific to said another analyte is fixed to said another test line.

17. A device according to claim 16, wherein more than one analytes are selected from the dry eye-specific biomarker group consisting of LTα, IL-4, IL-13, GM-CSF, IL-3, IL-5, IL-10, IL-9, and/or any derivative, fragment or precursor of any of the foregoing.

18. A device according to claim 16, wherein the one analyte is LTα and said another analyte is IL-4.

19. A lateral flow immunoassay device for diagnosing or monitoring ocular surface inflammation or dry eye disease in a subject, a predisposition thereto, or monitoring efficacy of therapy therefor, comprising: a base member and a horizontal array disposed on said base member, the horizontal array comprising:
(i) a conjugate pad disposed on one end of the base member, comprising a diffusively bound conjugate that comprises a detection reagent, optionally an antibody or antigen-binding antibody fragment, which detection reagent specifically binds a first immuno-complex with a dry eye biomarker, if present, in a tear sample added to the conjugate pad, the conjugate also comprising a and a label; and
(ii) a wicking membrane contact with the conjugate pad to receive the tear sample from the conjugate pad and comprising a capture reagent that is immobilized in a test line of the wicking membrane, is specific to the biomarker, and which can combine with the first immuno-complex to form a second immuno-complex fixed to the test line; and
(iii) the wicking membrane further comprises a third binder which does not bind to the biomarker but binds to the detection reagent and is immobilized in a control line of the wicking membrane, the control line being located downstream of the test line; and
(iv) an absorbent pad disposed on the other end of the base member and being in contact with the wicking membrane.

20. A device according to claim 19, wherein the conjugate pad further comprises a tear sample receiving area and a buffer receiving area, the tear sample receiving area being downstream of the buffer receiving area.
